(19) United States
(12) Patent Application Publication
(10) Pub. No.: US 2026/0207475 A1
(43) Pub. Date: Jul. 23, 2026

(54) Multi-Phase Transdermal Enhancer System for Targeted Dermal and Sub-dermal and Musculoskeletal Delivery

(71) Applicant: Thomas Rhee, Tampa, FL (US)

(72) Inventor: Thomas Rhee, Tampa, FL (US)

(21) Appl. No.: 19/566,847

(22) Filed: Mar. 13, 2026

Related U.S. Application Data
(60) Provisional application No. 63/905,320, filed on Oct. 24, 2025.

(51) Int. Cl. A61K 9/00; A61K 9/06; A61K 31/045; A61K 31/165; A61K 47/14; A61K 47/18

(52) U.S. Cl. CPC A61K 9/0014; A61K 9/06; A61K 31/045; A61K 31/165; A61K 47/14; A61K 47/18

(57) ABSTRACT

The present invention relates to a multi-phase transdermal enhancer system designed to increase the penetration and bioavailability of active ingredients through the epidermal barrier while maintaining skin integrity and minimizing irritation. The system combines a synergistic blend of solvents, co-solvents, humectants, and fatty-acid modifiers that work cooperatively to transiently alter lipid packing in the stratum corneum, enhance solute partitioning, and improve diffusion kinetics. The composition is suitable for incorporation into cosmetic, therapeutic, and over-the-counter topical formulations, enabling deep, controlled, and sustained delivery of both hydrophilic and lipophilic actives. The invention further provides formulations, methods of use, and benefits such as improved stability, enhanced absorption efficiency, and compatibility with diverse bioactive agents.

### FIELD OF THE INVENTION

The present invention relates to a multi-phase transdermal enhancer system designed for targeted dermal, sub-dermal, and musculoskeletal delivery of active agents.

### CROSS-REFERENCE TO RELATED APPLICATIONS

This application is a continuation of U.S. patent application Ser. No. 63/905,320 filed Oct. 24, 2025, the disclosures of which are hereby incorporated by reference in its entirety.

### BACKGROUND

Topical formulations are often constrained by the skin's natural barrier function, primarily the stratum corneum, which significantly limits the absorption of active ingredients. Conventional penetration enhancers frequently fall short due to inefficiency, instability, or irritation at concentrations necessary for effective delivery. Consequently, there remains a critical demand for a safe, consistent, and versatile system capable of transporting bioactive molecules across multiple skin layers while preserving epidermal integrity and maintaining formulation stability. Most existing delivery vehicles rely predominantly on either volatile solvents or occlusive lipids, yet few successfully integrate a balanced mechanism that combines volatility-driven diffusion, polarity modulation, and lipid-phase fluidization. The present system overcomes these challenges through a distinct multi-phase architecture engineered to facilitate controlled, biocompatible molecular transport and optimized active delivery performance.

Although individual penetration enhancers such as short-chain alcohols, fatty acids, glycol ethers, and humectants are known in the art, such systems generally operate through singular or additive mechanisms, often relying on aggressive lipid disruption, solvent extraction, or occlusive hydration. These conventional approaches frequently produce transient enhancement accompanied by irritation, uncontrolled evaporation, or prolonged barrier compromise. In contrast, the present invention is directed to a structurally coordinated, multi-phase dermal modulation architecture in which volatility-driven activation, reversible lipid lamellae fluidization, polarity-gradient transport, and hydration-regulated retention occur in a controlled and sequential manner. The inventive system therefore differs from conventional enhancer mixtures by producing non-additive, phase-dependent transport behavior and controlled reversibility not achieved by individual enhancer components used alone or in arbitrary combination.

### SUMMARY

The present invention discloses a novel transdermal enhancer system composed of a synergistic blend of solvents, co-solvents, humectants, and fatty acid-based permeability modifiers. This optimized combination enables deep dermal penetration and precise delivery of active compounds, including botanical cell-free bioactive biomolecules, peptides, analgesics, enzymes, antioxidants, and regenerative agents. The system operates through a multi-phase diffusion mechanism that balances volatile and non-volatile carriers to control evaporation rate and diffusion flux. Its polarity gradient is carefully optimized to ensure miscibility between hydrophilic and lipophilic actives, thereby improving solubilization and partitioning efficiency. The formulation provides barrier-safe enhancement through temporary and reversible lipid disruption, increasing permeability without compromising the protective integrity of the stratum corneum. Broadly compatible with cosmetic, therapeutic, and regenerative applications, the system also supports controlled delivery characterized by enhanced penetration depth and a sustained release profile, as demonstrated in both in-vitro and ex-vivo diffusion models. Overall, this invention enables consistent and efficient transport of active ingredients to targeted tissue layers, resulting in superior absorption efficiency, faster onset, and prolonged duration of action compared to conventional topical delivery systems.

### BRIEF DESCRIPTION OF THE DRAWINGS

FIG. 1 shows cross-sectional schematic of the skin and sub-dermal tissues showing solvent-driven diffusion through the stratum corneum, epidermis, dermis, and into subcutaneous and muscle layers, and the polarity gradient.

FIG. 2 shows graph comparing diffusion rate (flux) versus time between control and enhanced formulations.

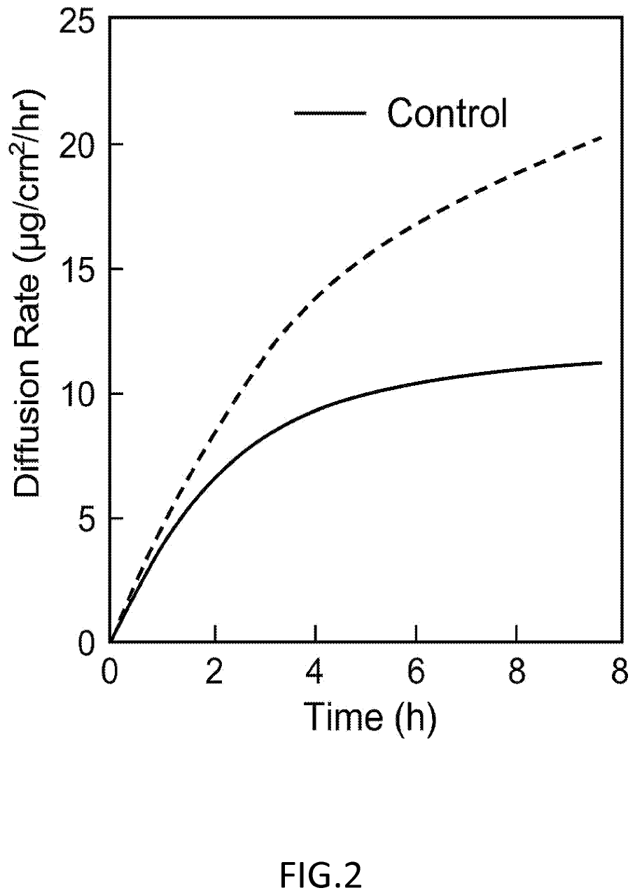

FIG. 3 shows structural diagram of formulation phases—volatile solvent layer, lipid modifier layer, and humectant-retention phase—demonstrating multi-phases diffusion behavior.

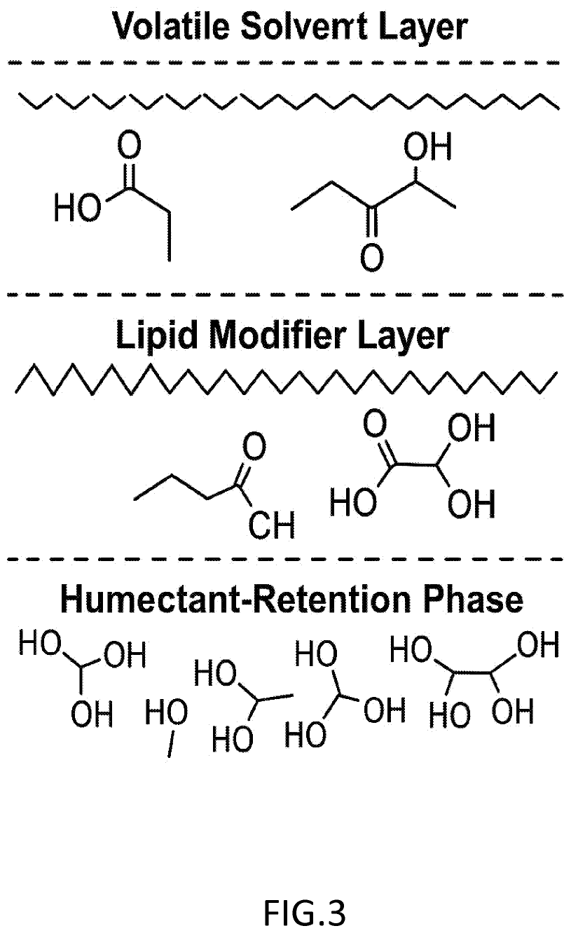

### DETAILED DESCRIPTION

It is to be understood that the aspects of the present invention as described below are not limited to specific compositions, methods or preparing such compositions, or uses thereof, as such may, of course, vary. It is also to be understood that the terminology used herein is for the purpose of describing particular aspects only and is not intended to be limiting.

The present invention relates to a proprietary multi-phase transdermal enhancer system designed for targeted dermal, sub-dermal, and musculoskeletal delivery of active agents. The system facilitates deep, sustained, and controlled penetration of active ingredients while maintaining epidermal integrity. It is suitable for incorporation into both cosmetic and therapeutic topical formulations, providing enhanced absorption efficiency, improved formulation stability, and minimized potential for irritation.

The enhancer system operates through the establishment of a dynamic polarity gradient and transient lipid phase fluidization within the stratum corneum, thereby promoting diffusion of active compounds across multiple skin layers without disrupting the natural barrier function. The composition is modular, customizable, and compatible with a wide range of bioactive molecules, enabling versatile formulation design and targeted delivery profiles.

For purposes of this disclosure, the terms “reversible,” “temporary,” and “without permanent structural disruption” refer to modulation of stratum corneum proteins and intercellular lipid lamellae that returns substantially to baseline physiological barrier function following evaporation or dissipation of the volatile fraction and equilibration of non-volatile components. Reversibility may be characterized by normalization of transepidermal water loss (TEWL), corneometry measurements, or barrier impedance within a clinically acceptable recovery window, typically within approximately 2 to 24 hours following application, without histologically detectable lipid extraction, persistent barrier damage, or cytotoxic alteration of epidermal architecture. The modulation described herein therefore differs from destructive solvent systems or keratolytic disruption mechanisms that result in permanent lipid removal or sustained barrier compromise.

### Composition of the Invention:

The primary solvent system facilitates the solubilization and initial penetration of active ingredients through the epidermis. Common solvents used for this purpose include ethanol (3-15% w/w), which enhances penetration efficiency, and optionally isopropyl alcohol (0.1-1% w/w) for additional solubilization support. Diethylene glycol monoethyl ether (DEGEE) at concentrations of 5-20% w/w is also frequently employed due to its excellent ability to transport actives through the skin barrier.

Co-solvents serve to promote miscibility between polar and nonpolar components, thereby ensuring compatibility with both hydrophilic and lipophilic actives. Typical examples include propylene glycol (2-10% w/w), which functions as both a solvent and a humectant, and dimethyl isosorbide (1-5% w/w), known for its role in enhancing solubility and delivery of actives. Butylene glycol may also be incorporated optionally at 1-5% to further improve texture and stability.

As used herein, an “amount sufficient” to induce the described dermal modulation refers to a concentration of a given component that produces reversible enhancement of stratum corneum permeability without permanent structural disruption or lipid extraction, as described in this specification. In some embodiments, such amounts correspond to concentrations within the ranges specified for each component herein, and in other embodiments may include other concentrations capable of producing reversible barrier modulation with subsequent recovery of physiological barrier function.

Lipid-phase enhancers act by transiently altering the organization of intercellular lipids to increase skin permeability while supporting barrier recovery after application. Representative examples include oleic acid (1-8% w/w), which fluidizes the lipid matrix, and isopropyl myristate (0.5-5%), a classic penetration enhancer with emollient properties. Medium-chain triglycerides (0.5-3%) can also be added to optimize the balance between enhancement efficacy and skin compatibility.

Humectants and stabilizers are incorporated to maintain skin hydration, improve formulation stability, and reduce the potential for irritation. Common components include urea (0.5-5% w/w) for moisture retention, glycerin (1-5%) as a widely used humectant, and panthenol (0.1-1%) for its soothing and regenerative properties. Tocopherol (vitamin E) may also be included as an optional antioxidant stabilizer to enhance product longevity and protect sensitive ingredients from oxidation.

### Mechanism of Action

The transdermal enhancer system configured to facilitate the transport of one or more active compounds from a topical formulation across the epidermal and dermal layers and into subcutaneous and muscular tissues. The mechanism of action comprises a coordinated, multi-phase diffusion process, each phase performing a defined and synergistic function, namely: surface activation, barrier modulation, polarity-driven migration, and controlled retention. The combined operation of these four phases enables efficient and sustained delivery of actives while maintaining the physiological and structural integrity of the epidermal barrier.

### Phase 1—Volatile Solvent Activation (Surface Opening)

In one embodiment, the first phase comprises a volatile solvent activation step wherein one or more volatile solvents are employed to hydrate and transiently plasticize the stratum corneum. The volatile solvent component may comprise ethanol, isopropanol, or equivalent low-molecular-weight alcohols or esters. Upon topical application, rapid evaporation of said volatile solvents at physiological skin temperature results in a decrease in surface tension and the generation of a localized thermodynamic gradient.

This gradient drives solvent flux in a downward direction, thereby facilitating the penetration of actives into the superficial epidermal layers. The transient hydration effect softens corneocyte junctions and produces microchannels within the lipid matrix, establishing an initial diffusion force along polarity and solubility gradients. Accordingly, the volatile fraction functions as a surface activation agent, temporarily opening the outermost skin barrier and enabling subsequent migration of co-solvent and lipid-phase enhancers.

### Phase 2—Lipid Disruption and Fluidization (Barrier Modulation)

In some embodiments, the second phase comprises the incorporation of lipid-phase enhancers that act to modulate and fluidize the lipid lamellae of the epidermal and upper dermal regions. Suitable lipid-phase enhancers include, but are not limited to, oleic acid, isopropyl myristate, and medium-chain triglycerides.

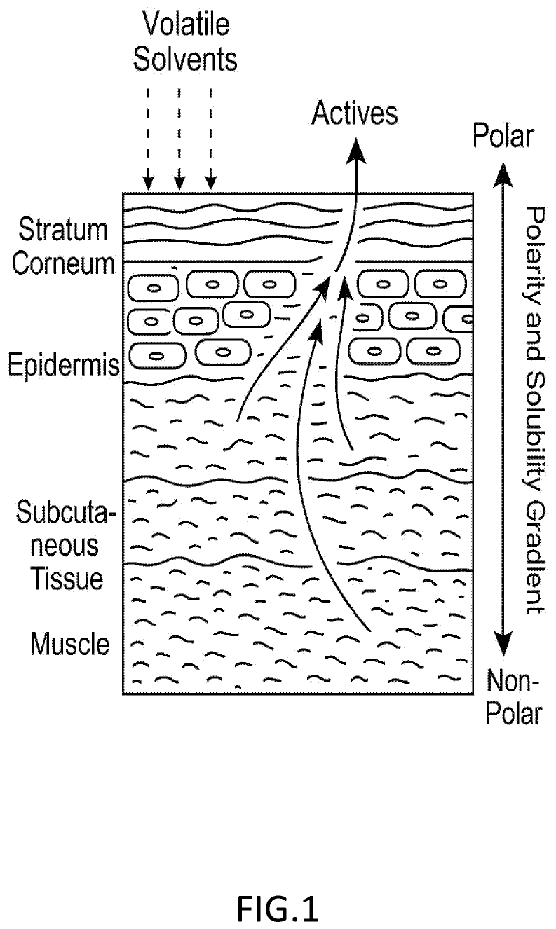

These amphiphilic agents are operative to intercalate within intercellular lipid bilayers, inducing reversible lipid fluidization and enhancing permeability without causing permanent disruption or cytotoxicity. The temporary rearrangement of lipid domains permits concurrent transport of both hydrophilic and lipophilic active species. As depicted in FIG. 1, this phase corresponds to a mid-layer diffusion zone wherein polarity transitions from a highly polar surface region to a moderately non-polar dermal region, thereby optimizing the migration of solutes through the lipid matrix.

### Phase 3—Polarity Gradient Migration (Deep Transport)

In another embodiment, the third phase establishes a polarity and solubility gradient extending from the epidermal surface into deeper dermal and subdermal layers. The system may comprise one or more co-solvents selected from diethylene glycol monoethyl ether (DEGEE), propylene glycol, and dimethyl isosorbide, optionally combined with humectants such as glycerin or urea.

Said combination forms a continuous diffusion bridge between hydrophilic and lipophilic domains. Upon dissipation of the volatile fraction, the non-volatile carriers maintain a concentration gradient that propels active molecules through both aqueous and lipidic pathways. Osmotic pressure differentials and hydrogen-bonding interactions between the carrier matrix and epidermal proteins further reinforce the diffusion process, thereby facilitating sustained, polarity-driven migration of active compounds into deeper subdermal and muscular tissues.

### Phase 4—Controlled Retention (Sustained Release)

In further embodiments, the fourth phase provides a controlled

retention mechanism configured to prolong dermal residence and release of active compounds. The retention phase comprises non-volatile components including, but not limited to, glycols, humectants, and lipid stabilizers. These materials function as localized reservoirs within dermal and subdermal tissue interfaces, thereby maintaining hydration and retarding evaporation.

The enhanced performance of the present system is attributable to the coordinated temporal interaction of its defined phases rather than the isolated action of any single penetration enhancer. The volatile solvent fraction establishes an initial thermodynamic and concentration gradient; the lipid-phase modulators transiently reorganize intercellular lipid domains in a reversible manner; the polarity-gradient establishing co-solvents sustain directional migration across heterogeneous dermal environments; and the hydration-regulating components stabilize barrier function while maintaining diffusion flux. This structured phase progression results in a diffusion profile characterized by reduced lag time, increased percutaneous flux, and controlled dermal retention relative to compositions lacking coordinated volatility and polarity modulation. Such non-additive and sequentially dependent behavior represents an improvement over conventional enhancer systems and would not be predicted based solely on the known properties of the individual components.

Said components preserve a residual concentration gradient conducive to gradual, time-dependent release of actives. The controlled retention mechanism enhances bioavailability and therapeutic duration while mitigating rebound evaporation, irritation, and excessive transepidermal water loss. The combined operation of all four phases produces a balanced, sustained-delivery system resulting in measurable accumulation of active ingredients within subcutaneous and muscular structures, consistent with the diffusion profile illustrated in FIG. 1.

The enhancer system described herein provides multiple functional and formulation-related advantages over conventional transdermal delivery systems. The multi-phase diffusion mechanism enables deeper and more controlled penetration of active compounds through the epidermal and dermal barriers into subcutaneous and muscular tissues, thereby enhancing therapeutic efficacy and bioavailability. The system maintains the physiological safety and hydration of the skin barrier, preventing excessive trans-epidermal water loss or irritation commonly associated with aggressive penetration enhancers.

Furthermore, the composition is compatible with a wide range of active substances, including botanical extracts, peptides, enzymes, small-molecule actives, and other bioactive agents, making it versatile for cosmetic, dermatological, and pharmaceutical applications. The synergistic solvent-humectant-lipid system provides formulation stability and extended shelf life by minimizing phase separation and oxidative degradation. In addition, the balanced volatility profile reduces evaporation-driven loss of actives and minimizes skin irritation, resulting in a controlled, safe, and efficient transdermal delivery system.

### Example Formulation

| Component | Function | % w/w |
|---|---|---|
| Ethanol | Primary solvent | 10.0 |
| DEGEE/Diethylene Glycol | ) Solvent enhancer | 12.0 |
| Monoethyl Ether | | |
| Propylene Glycol | Co-solvent | 5.0 |
| Dimethyl Isosorbide | Co-solvent | 3.0 |
| Oleic Acid | Lipid-phase enhancer | 3.0 |
| Urea | Humectant | 2.0 |
| Panthenol | Skin conditioner | 0.5 |
| Vitamin E (Tocopherol) | Antioxidant | 0.3 |
| Purified Water | Balance to 100% | |

An exemplary embodiment of the invention comprises a topical analgesic or regenerative cream formulated with the described enhancer system to optimize the delivery of bioactive agents such as menthol, capsaicin, peptides, plant-derived exosome analogs, and other lipid-soluble botanical compounds.

In performance evaluations, formulations incorporating this system demonstrated significantly enhanced percutaneous flux rates in in-vitro permeation testing (IVPT) models, along with a shorter lag time for active onset compared to control formulations. The system also maintained sustained skin hydration with minimal irritation following repeated applications and provided improved solubility, uniform dispersion, and enhanced stability of dual-phase actives within the emulsion matrix.

The enhancer system demonstrates robust stability across a broad pH range (4.5-7.5) and is compatible with multiple product types, including creams, serums, gels, and emulsions.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims.

What is claimed is:

1. A reversible multi-phase dermal modulation system configured to enhance transdermal transport of at least one bioactive compound, comprising:
a surface-modulating component configured to induce temporary and reversible plasticization of stratum corneum proteins without permanent barrier disruption;
a lipid-structure modulating component configured to reversibly fluidize intercellular lipid lamellae within the stratum corneum without lipid extraction or irreversible structural damage;
a polarity-gradient establishing transport component configured to create a transient polarity gradient across the stratum corneum and viable epidermis to facilitate sequential partitioning of the bioactive compound through hydrophilic and lipophilic microdomains; and
a hydration-regulating component configured to maintain controlled dermal hydration and osmotic gradients sufficient to sustain diffusion into deeper dermal tissue;
wherein the components function sequentially and synergistically to produce controlled, reversible modulation of dermal barrier properties.

2. The system of claim 1, wherein the surface-modulating component comprises ethanol, isopropanol, or combinations thereof.

3. The system of claim 1, wherein the lipid-structure modulating component comprises oleic acid, isopropyl myristate, a fatty acid ester, or combinations thereof.

4. The system of claim 1, wherein the polarity-gradient establishing transport component comprises diethylene glycol monoethyl ether, dimethyl isosorbide, or combinations thereof.

5. The system of claim 1, wherein the hydration-regulating component comprises glycerin, urea, propylene glycol, butylene glycol, or combinations thereof.

6. The system of claim 1, wherein the surface-modulating component is present at about 0.1% to about 20% by weight.

7. The system of claim 1, wherein the lipid-structure modulating component is present at about 0.1% to about 10% by weight.

8. The system of claim 1, wherein the polarity-gradient establishing transport component is present at about 1% to about 15% by weight.

9. The system of claim 1, wherein the hydration-regulating component is present at about 0.1% to about 15% by weight.

10. The system of claim 1, wherein the transdermal composition facilitates delivery of one or more bioactive compounds to dermal and subdermal tissues including dermis, subcutaneous tissue, fascia, periarticular tissue, and underlying musculoskeletal structures.

11. The system of claim 1, wherein the volatile solvent, lipid-phase modulating agent, polarity-gradient establishing co-solvent, and hydration-regulating component are present in relative proportions sufficient to produce sequential, phase-dependent modulation of dermal barrier properties resulting in enhanced percutaneous flux with reversible barrier recovery.

12. A method for enhancing transdermal delivery of a bioactive compound comprising a composition comprising:
a surface-modulating component configured to induce temporary and reversible plasticization of stratum corneum proteins without permanent barrier disruption;
a lipid-structure modulating component configured to reversibly fluidize intercellular lipid lamellae within the stratum corneum without lipid extraction or irreversible structural damage;
a polarity-gradient establishing transport component configured to create a transient polarity gradient across the stratum corneum and viable epidermis to facilitate sequential partitioning of the bioactive compound through hydrophilic and lipophilic microdomains; and
a hydration-regulating component configured to maintain controlled dermal hydration and osmotic gradients sufficient to sustain diffusion into deeper dermal tissue;
wherein the components function sequentially and synergistically to produce controlled, reversible modulation of dermal barrier properties.

13. The method of claim 12, wherein the surface-modulating component comprises ethanol, isopropanol, or combinations thereof.

14. The method of claim 12, wherein the lipid-structure modulating component comprises oleic acid, isopropyl myristate, a fatty acid ester, or combinations thereof.

15. The method of claim 12, wherein the polarity-gradient establishing transport component comprises diethylene glycol monoethyl ether, dimethyl isosorbide, or combinations thereof.

16. The method of claim 12, wherein the hydration-regulating component comprises glycerin, urea, propylene glycol, butylene glycol, or combinations thereof.

17. The method of claim 12, wherein the surface-modulating component is present at about 0.1% to about 20% by weight.

18. The method of claim 12, wherein the lipid-structure modulating component is present at about 0.1% to about 10% by weight.

19. The method of claim 12, wherein the polarity-gradient establishing transport component is present at about 1% to about 15% by weight.

20. The method of claim 12, wherein the hydration-regulating component is present at about 0.1% to about 15% by weight.

21. The method of claim 12, wherein the transdermal composition is a semi-solid selected from the group consisting of a gel, a lotion, a cream, an ointment, a serum, and a foam.

22. The method of claim 12, wherein the transdermal composition facilitates delivery of one or more bioactive compounds to one or more tissues including but not limited to dermis, subcutaneous tissue, fascia, skeletal muscle, tendons, ligaments, and periarticular tissue.
